(19) United States
(12) Patent Application Publication
(10) Pub. No.: US 2016/0347739 A1
(43) Pub. Date: Dec. 1, 2016

(54) 5-OXOPYRROLIDINE DERIVATIVES AS HIV INTEGRASE INHIBITORS

(71) Applicant: Xiang Simon WANG, Washington, DC (US)

(72) Inventor: Xiang Simon WANG, Washington, DC (US)

(73) Assignee: Howard University, Washington, DC (US)

(21) Appl. No.: 15/220,049

(22) Filed: Jul. 26, 2016

Related U.S. Application Data
(63) Continuation-in-part of application No. PCT/US2015/017810, filed on Feb. 26, 2015.
(60) Provisional application No. 61/944,981, filed on Feb. 26, 2014.

(51) Int. Cl. C07D 403/06

(52) U.S. Cl. CPC C07D 403/06

(57) ABSTRACT

A method for treating against HIV, such as by inhibiting HIV integrase, in target cells or in a patient involves administering to target cells or to a patient in need of treatment an effective amount of at least one compound having an N-indol heteroarylcarboxamide scaffold which compound is represented by the formula:

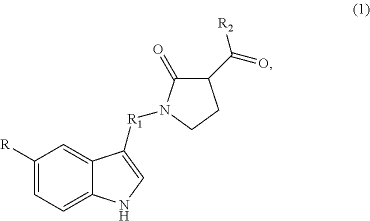

wherein, independent of each other,
R independently represents hydrocarbyl, halogeno, amino, substituted amino, or alkoxy,
R₁ represents di-valent hydrocarbyl, substituted or unsubstituted, and
R₂ represents an ether moiety.

### RELATED APPLICATIONS

This application is a continuation in part of PCT/US2015/17810, filed Feb. 26, 2015, which designated the United States, and claims priority to U.S. Provisional Application 61/944,981, filed Feb. 26, 2014, the complete disclosures of which are incorporated by reference.

### GOVERNMENT INTEREST

No government support was granted for this work.

### FIELD OF INVENTION

This work has identified novel compounds with antiviral activity, specifically novel integrase inhibitors for the treatment of HIV. By inhibiting nuclear import of HIV integrase with small molecule inhibitors, integration of HIV viral genomic DNA into host DNA has been prevented thereby blocking HIV replication.

### BACKGROUND

The human immunodeficiency virus (HIV), as its name suggest, is characterized by progressive immunologic deterioration which over a period of time results in neurologic disorders and opportunistic infections leading to acquired immunodeficiency syndrome (AIDS). The search to find antiretroviral therapy for treatment of the 34 million people globally infected with Human Immunodeficiency Virus (HIV) is an ongoing one. Although there are over 20 antiretroviral drugs approved for the treatment of HIV that will halt replication of the virus, the complete eradication of this fatal disease remains a scientific challenge. Drug resistance, tolerability and HIV latency are major factors contributing to ART failure and ultimately success in finding a cure. Hence, there remains a critical and unmet need to identify novel antiretroviral drug candidates active against HIV-1 resistant mutations for treatment of HIV-1 infection.

Integrase is one of the essential enzymes required for replication of HIV and is encoded by viral pol gene. Integration of virally transcribed cDNA into host DNA is an essential step for viral replication and the continuation of HIV life cycle. Upon synthesis of viral DNA in the cytoplasm of the cell, a series of interactions of viral proteins, matrix protein, a triple stranded cDNA flap and cellular cofactors, with integrase (IN) forms the pre-integration complex (PIC). Transport of viral DNA to the nucleus of the cell requires the formation of PIC which binds nuclear transport receptors via a nuclear localization signal (NLS) thereby allowing for entry into the nucleus where viral cDNA will integrate into host DNA. Successful integration of viral cDNA into host DNA is an essential step for viral replication and the continuation of HIV life cycle hence, IN is considered a good drug target for the development of ARV drugs.

There are multiple classes of antiretroviral (ARV) drugs that target various stages of the HIV life cycle which elicit unique mechanisms of action. As the mechanism of action of integrase (IN) and the evolution of IN resistant mutations is swiftly unfolding, IN represents an untapped source of undiscovered ARV drugs. The success rate for discovery and development of integrase inhibitors is quite low with only two IN inhibitors currently on the market, Raltegravir (RAL) developed by Merck & Co and Elvitegravir (EVG), as a combination therapy, developed by Gilead Science. Both drugs binds the catalytic site of IN located in the catalytic core domain and function by inhibiting the strand transfer process of vDNA into host DNA hence are referred to as integrase strand transfer inhibitors (INSTI). However due to the increasing clinical reports of INSTI resistance there is a need to design new class of IN inhibitors with novel mechanism of action. Specifically RAL and EVG exhibit consistant resistant pathways Q148HRQ/G140S and N155H/E92Q. A more recent approach to targeting IN is with small molecule inhibitors of LEDGF/p75 known as LEDGINs. Integrase inhibitors have been classified into five categories: (1) DNA-binding inhibitors, (2) 3′ processing inhibitors, (3) nuclear translocation/import inhibitors, (4) strand transfer inhibitors, and (5) gap repair inhibitors. Currently there are no reports of small molecule inhibitors that target allosteric site on CTD of IN.

The structure of IN plays a major role in dictating its function hence, complete elucidation of its structure will contribute significantly to the discovery of integrase inhibitors. To date the complete crystal structure of IN has not been elucidated, however we do know IN comprises 288 amino and has three domains, N-terminal domain (NTD), catalytic core domain (CCD) and the C-terminal domain (CTD). Each IN monomer will combine to form a tetrameric IN structure. The NTD and CCD have conserved and functional motifs, while the CTD is the least conserved of the three. The conserved region of the NTD (residues 1-50) contains a sequence of HHCC residues that form a zinc finger motif which functions to chelate one zinc atom per IN monomer. In the absence of zinc the NTD of IN is destabilized and become disordered and formation of the multimeric form of IN is not achieved and could disrupt its activity. The CCD (residues 51-212) conserved region comprises a triad of acidic residue that form the DDE motif It is essential for 3′ processing and strand transfer processes. Integrase has nuclease activity that is site specific for cleaving two nucleosides at the 3′ end of viral DNA, a process known as 3′ processing. Subsequently, the strand transfer process ensues and it involves the 3 ends of viral DNA inserting into host DNA. The CTD (residues 213-288) on the other hand, is less conserved and is essential for IN-IN and non-specific IN-DNA (residues 220-270) interactions.

### SUMMARY

Methods for treating against HIV-1 and for inhibiting HIV integrase in target cells or in a patient in need of treatment include administering certain anti-viral compounds having an N-indol heteroacrylcarboxamide scaffold for retroviral therapy including but not limited to treating against HIV-1 and for inhibiting HIV integrase are described.

The present invention demonstrates specificity of the molecules to bind to and inhibit HIV integrase activity as well as their antiviral activity against HIV-1, such as the HIV-1 lentiviral vector. Hence these compounds include exhibit a novel mechanism of action against the HIV-1 virus and HIV integrase (IN).

A class of small molecules is identified as HIV integrase (IN) inhibitors that bind to site(s) targeting IN residue(s) so as to effectively inhibit HIV replication. Small molecules can bind to inhibit such residue(s). A compound that binds to allosteric sites for residues K236/K240 and R262/R263/K266 includes an exemplary compound as described herein, such as compound 209.

### BRIEF DESCRIPTION OF THE FIGURES

FIG. 1 shows screening assay results for C-terminal domain of HIV-1 IN, nuclear import inhibition in which

FIG. 1(A) shows data for the positive control, eGFP-MBP-IN-WT with no drug results in nuclear localization of IN;

FIG. 1(B) shows data for the negative control, eGFP-MBP-IN-R262/3/6A chimeric triple mutant exhibiting nuclear exclusion of the fusion protein;

FIG. 1(C) shows data for test compound (“209”) with eGFP-MBP-IN-WT exhibiting similar phenotype as that of eGFP-MBP-IN-R262/3/6A that is, nuclear exclusion of the fusion protein; and

FIG. 1 (D) shows data for a test compound (“535”) with eGFP-MBP-IN-WT exhibiting again nuclear exclusion of the fusion protein in majority of cells.

FIG. 2 is a dose response curve of an exemplary test compound and AZT.

### DETAILED DESCRIPTION

A method for treating for inhibition of HIV infection comprises administering to an infected cell(s) or a patient in need of treatment an effective amount of at least one anti-viral compound, wherein the anti-viral compound comprises a compound having an N-indol heteroarylcarboxamide scaffold represented by the formula (1):

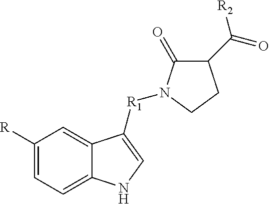

wherein, independent of each other,

R represents hydrocarbyl, halogeno, amino, substituted amino, or alkoxy,

R₁ a di-valent hydrocarbyl, substituted or unsubstituted, such as di-valent aryl or divalent alkyl, and

R₂ represents an ether moiety —R₅—O—R₆, wherein R₅ and R₆ are independently alkyl or aryl.

While R is shown in one position in formula (1), the R group can be at any other position on the ring.

In another of its aspects, a method for treating retroviral infections and more specifically inhibition of HIV infection comprises administering to an infected cell(s) or a patient in need of treatment an effective amount of at least one anti-viral compound, wherein the anti-viral compound comprises a compound represented by the formula (1) in which R represents alkyl.

In another of its aspects, a method for treating retroviral infections and more specifically inhibition of HIV infection comprises administering to an infected cell(s) or a patient in need of treatment an effective amount of at least one anti-viral compound, wherein the anti-viral compound comprises a compound represented by the formula (1) in which R represents alkoxy.

In another of its aspects, a method for treating retroviral infections and more specifically inhibition of HIV infection comprises administering to an infected cell(s) or a patient in need of treatment an effective amount of at least one anti-viral compound, wherein the anti-viral compound comprises a compound represented by the formula (1) in which R represents halogen.

In another of its aspects, a method for treating retroviral infections and more specifically inhibition of HIV infection comprises administering to an infected cell(s) or a patient in need of treatment an effective amount of at least one anti-viral compound, wherein the anti-viral compound comprises a compound represented by the formula (1) in which R represents amino or represents substituted amino that is represented by —NR₃,R₄ wherein R₃ and R₄, are not both hydrogen and independently represent alkyl or alkenyl.

In another aspect, a method for treating retroviral infections and more specifically inhibition of HIV infection comprises administering to an infected cell(s) or a human or mammal in need of treatment an effective amount of at least one anti-viral compound, in which at least one of the anti-viral compounds is N-(3-ethoxy propyl)-1-[2-(5-methyl-1H-indol-3-yl)ethyl]-5-oxopyrrolidine (“209”).

In another aspect, a method for inhibiting HIV-1 integrase in a cell(s) or in a patient comprises administering to a infected cell(s) or a patient in need of a treatment an effective amount of at least one HIV-1 integrase inhibitor comprising as least one compound represented by a formula or as described herein.

In another embodiment, the methods described can be administered as a single or combination therapy with one or more antiretroviral agents for the treatment of retroviral infections including but not limited to HIV infection in humans and mammals. Combination therapy can also include gene therapy.

In another embodiment, a pharmaceutical composition for treating retroviral infections (and inhibiting HIV-1 integrase) comprises at least one compound represented by formula (1) herein as an active ingredient. The pharmaceutical composition will include additional ingredient(s), such as adjuvant(s), as described elsewhere herein.

The compounds represented by the formulas herein may exist in tautomeric or resonance forms. All the tautomeric, resonance and isomeric forms are within the scope of the inventions herein.

The compounds represented by formulas herein can be in a pharmaceutically effective salt or, in principle, other pharmaceutically acceptable forms, such as an ester form, and the methods described herein can be practiced with the salt form or such other forms such compound(s). Pharmaceutical forms include those formed, as the case may be, with acetic acid, hydrobromic acid, acetic acid, trifluoroacetic acid, citric acid, oxalic acid, benzoic acid, benzenesulfonic acid, toluenesulfonic acid, sulfuric acid, tartaric acid, fumaric acid, maleic acid, malic acid, lactic acid, and methanesulfonic acid, as examples, although this list is not intended to be limiting insofar as this description is concerned.

The compound(s) and/or their other pharmaceutically acceptable forms (salts as an example) can be administered orally in solid dosage forms, such as capsules, tablets and powders, or in liquid dosage forms, such as elixirs, syrups and suspensions, or injectable forms. It will be appreciated that administration to a cell(s) infected with HIV may be by a different route than administration to a patient. In principle, specialized electrostatic spray apparatus may be used.

It will be appreciated, that a method as described herein can further comprise identifying and selecting a suitable compound, such as a compound represented by formula (1), by conducting a suitable assay(s), such as an HIV-1 assay and/or an integrase assay, and thereafter administering the identified and selected compound to infected cells or to a patient in need of treatment.

It should be understood that the method of using compounds according to any of the formulas herein to treat HIV-1 or to inhibit HIV integrase are novel.

A small molecule inhibitor herein includes a compound represented by a formula herein that binds sites targeting residues of HIV integrase (sometimes referred to as IN or integrase herein) to inhibit HIV integrase (IN). Such compounds within a formula herein include those that bind to a site for at least one of the residues 1209, Pro238, Gla221, ASN222, ARG224, LYS240, ASP253, ASN254, K236, K240, K244, R262, R263, LYS266, ILE267, ARG269, and K266, preferably at least two of such residues. As an example, a compound within the compounds represented by formula (1) can bind sites targeting Lys240 and Pro 238 is an aspect of the present inventions.

In the above regards, although the CTD is less conserved than that of the NTD and CCD in IN, mutagenesis experiments have identified specific regions in the CTD that are highly conserved. Although CCD on its own can carry out reversal of DNA-strand transfer reaction in vitro, without the NTD and CTD catalysis of 3′ processing and strand transfer is impossible.

Mutations made in and around this region, which include residues K236/K240 and R262/R263/K266, resulted in loss of integrase activity and inhibited HIV replication as result of delocalization of IN outside the nucleus of cells. It is postulated that one of two things are happening, possibly a significant conformational change upon double or ripple mutations disrupts the SH3 like fold resulting in loss of interaction by nuclear transport receptors and viral proteins necessary for successful integration of vDNA. On the other hand, it is also possible the structural change could also result in loss of IN-DNA binding directly. However, until the present inventions, there has been no prior discovery of a small molecule(s) that binds to such residues (regions) of IN.

A compound(s) that binds two allosteric sites on the CTD or integrase and the methods for administering such compound(s) as disclosed herein are novel.

Accordingly, in one of its aspects, a method for treating against HIV-1 comprises administering a small molecule inhibitor that binds sites targeting residues in the C-terminal domain of HIV integrase. This includes a method for treating against HIV-1 comprises administering a small molecule inhibitor that binds allosteric sites targeting residues of the C-terminal domain of HIV integrase, with the administering being to target cells or to a patient in need of treatment. In one of its aspects, an embodiment of the invention concerns small molecule inhibitor(s) herein includes a compound within those represented by a formula herein that binds sites targeting residues in the C-terminal domain of HIV integrase, such as allosteric sites in the CTD or integrase, such as residues K236/K240 and R262/R263/K266, or that binds sites targeting such residues as Lys240 and Pro238, to mention examples.

In still another aspect, a method for treating against HIV-1 comprises administering a small molecule inhibitor that binds sites (targeting) at least one of residues Arg262, Arg263, Arg266, Lys244, Ile267, and Lys266 of HIV integrase, with the administering being to target cells or to a patient in need of treatment. The binding with Lys266 may depend on the binding orientation. A small molecule inhibitor herein includes a compound within those represented by formula herein that binds sites targeting such residue(s) of HIV integrase, preferably at least two of such residues.

In another aspect, a method for treating against HIV-1 comprises administering a small molecule inhibitor that binds sites (targeting) at least one of residues Arg262, Arg263 and Ile267 of HIV integrase, preferably at least two of the residues, with the administering being to target cells or to a patient in need of treatment. A small molecule inhibitor herein includes a compound within those represented by a formula herein that binds sites targeting such residues of HIV integrase, preferably at least two of such residues.

In one of its preferred aspects, a method for treating against HIV-1 comprises administering a small molecule inhibitor that binds sites (targeting) at least one of residues Lys240 and Pro238, Gln221, and Asp253 of HIV integrase, preferably at least two of the residues, with the administering being to target cells or to a patient in need of treatment. A small molecule inhibitor herein includes a compound within those represented by a formula herein that binds sites targeting such residues of HIV integrase, preferably at least two of such residues, such as Lys240 and Pro238.

In one of its aspects a method involves targeting sites on the CTD or integrase, in particular at least two allosteric sites on the CTD or integrase.

Representative compounds include compound 209, as an example.

In a further aspect, a compound(s) that is (are) novel and within the scope of the formulas herein forms part of the present inventions. The addition or inclusion of any provisos deemed appropriate to exclude any compound(s), specifically or generically, for any reason from any claim, such as a claim limited to the compounds per se, or from any formula is expressly reserved. Thus, a proviso that excludes non-novel compounds from a claim to the compounds per se is the meaning of the expression “A novel compound having an N-indol heteroarylcarboxamide scaffold selected from those represented by formula . . . ”

It will be appreciated that R can be hydrocarbyl, but it is not limited thereto. For instance, as disclosed herein, in another of its aspects, an R group can be halogen (sometimes referred to as halogeno), which includes, for example, bromo, chloro, fluoro, and iodo. It will be appreciated that R can be alkyl, including lower alkyl, or alkenyl, including lower alkenyl. R can be C₁-C₆ alkyl. Alkyl includes straight or branched alkyl. When R independently represents alkyl, such as a C₁-C₆ alkyl, exemplary alkyls include aliphatic and branched alkyls, such as methyl, ethyl, propyl, isopropyl, butyl, iso-butyl, pentyl and hexyl, as examples. R can be cycloalkyl with 3-6 carbon atoms in the cyclic portion, such as cyclopropyl, cyclopentyl and cyclohexyl, to mention examples. R can be C₂-C₆ alkenyl. In principle, there may be more than one R substituent. R is preferably lower alkyl, such as methyl, ethyl, propyl etc.

An R substituent can be an amino group (—NH₂) or and a substituted amino group (e.g., —NR₃R₄, when R₃ and R₄ are not both hydrogen). R₃ and R₄ independently can be alkyl, including lower alkyl, or alkenyl, including lower alkenyl. R₃ and R₄ can be different or identical. For instance, one of R₃ and R₄ can be alkenyl, such as a C₂-C₆ alkenyl, and the other can be alkyl. Each of R₃ and R₄ can be, independent of the other, a C₁-C₆ alkyl. Alkyl includes straight or branched alkyl. When R₃ and R₄ independently represent a C₁-C₆ alkyl, exemplary alkyls include aliphatic and branched alkyls, such as methyl, ethyl, propyl, isopropyl, butyl, iso-butyl, pentyl and hexyl, as examples. In principle, R₃ and/or R₄ can be cycloalkyl with 3-6 carbon atoms in the cyclic portion, such as cyclopropyl, cyclopentyl and cyclohexyl, to mention examples. At least one of R₃ and R₄ can be a C₂-C₆ alkenyl.

It will be appreciated that R includes alkoxy, including lower alkoxy, e.g., —OR₇ wherein R₇ is alkyl, including lower and cyclic alkyl, as discussed above. By way of example, —OR₇ includes methoxy, ethoxy, and propoxy (straight or branched).

It will be appreciated that R₁ can be a suitable di-valent aryl or alkylene group. For instance, when R₁ is alkylene, it can be lower alkylene. R₁ can be a divalent alkylene group having at least one carbon atom, such as from one to ten carbon atoms, including a C₁ to C₇ group by way of example. R₁ can be straight chain, branched, or cyclic as discussed above for R, but R₁ is by present preference an alkylene group, preferably straight chain. An alkylene group includes an aliphatic group, including —(CH₂)ₙ— with n being an integer of 1, 2, 3, 4, 5, 6 or 7, e.g., —(CH₂)₃— by way of example of an R group, but the methods and compounds are not necessarily so limited. When R₁ is di-valent alkylene or di-valent aryl it may or may not be substituted. R₁ can be aryl, such as phenylene as an example.

It will be appreciated that R₂ is an ether moiety that is represented by —R₅—O—R₆ wherein R₅ and R₆ are independent of one another. R₅ is a di-valent alkyl, aryl or cyclic alkyl group by way of example, and may be selected with reference to the divalent groups for R₁. R₅ can be a divalent alkylene group having at least one carbon atom, such as from one to ten carbon atoms, including a C₁ to C₇ group by way of example. R₅ can be straight chain, branched, or cyclic as discussed above for R, but R₃ is by present preference an alkylene group, preferably straight chain. An alkylene group for R₅ includes an aliphatic group, including —(CH₂)ₙ— with n being an integer of 1, 2, 3, 4, 5, 6 or 7, e.g., —(CH₂)₃— by way of example of an R₅ group, but the methods and compounds are not necessarily so limited. When R₅ is di-valent alkylene or di-valent aryl it may or may not be substituted. R₅ can be aryl, such as phenylene as an example. R₆ is an alkyl, alkenyl, aryl or cyclic alkyl group by way of example, and insofar as alkyl, alkenyl and cyclic alkyl are concerned they may be as selected with reference to R₃ and R₄. For instance, R₆ can be an alkyl having at least one carbon atom and includes straight or branched alkyl. R₆ includes lower alkyl, which includes C₁-C₆ alkyl. When R₆ represents a C₁-C₆ alkyl, exemplary alkyls include aliphatic and branched alkyls, such as methyl, ethyl, propyl, isopropyl, butyl, iso-butyl, pentyl and hexyl, as examples. In principle, R₆ can be cycloalkyl with 3-6 carbon atoms in the cyclic portion, such as cyclopropyl, cyclopentyl and cyclohexyl, to mention examples. R₆ can be alkenyl, which includes a C₂-C₆ alkenyl.

When R₅ and R₆ independently represent a C₁-C₆ alkyl, exemplary alkyls include aliphatic and branched alkyls, such as methyl, ethyl, propyl, isopropyl, butyl, iso-butyl, pentyl and hexyl, as examples. Each of R₅ and R₆ can be, independent of the other, a C₂-C₆ alkenyl.

In one of its aspects, the invention herein relates to the use of at least one compound as described herein as an active ingredient in a pharmaceutical composition, drug or medicament. Accordingly, a compound described is useful in making a drug for treating and/or the prevention of an HIV infection, and/or for reducing HIV replication, and/or for inhibiting HIV integrase (IN), and thus the methods described herein encompass the administration of such a product (drug etc.). A composition with such a compound(s) can include pharmaceutically acceptable carrier(s), vehicle(s) and diluent(s), which include those described in Gennaro et. al. (eds.), Remington, The Science and Practice of Pharmacy, (20^{th} Edition, 2000). The pharmaceutical composition can be formulated based on the mode of administering.

Administering includes orally, parenterally by inhalation spray, topically, rectally, nasally, buccally, vaginally or via an implanted reservoir. Oral administration or administration by injection may be preferable with some patients. The term “parenteral” as used herein includes subcutaneous, intracutaneous, intravenous, intramuscular, intra-articular, intrasynovial, intrasternal, intrathecal, intralesional and intracranial injection or infusion techniques.

For injection, such as a sterile injectable preparation, a sterile injectable aqueous or oleaginous suspension can be used. Such a suspension may be formulated using suitable pharmaceutically acceptable dispersing or wetting agents and suspending agents. The sterile injectable preparation may also be a sterile injectable solution or suspension in a non-toxic parenterally acceptable diluent or solvent. In addition, sterile, fixed oils are conventionally employed as a solvent or suspending medium. For this purpose, any bland fixed oil may be employed including synthetic mono- or diglycerides. For principle, fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant or a similar alcohol.

Orally administered includes any orally acceptable dosage form including, but not limited to, capsules, tablets, and aqueous suspension and solutions. In the case of tablets for oral use, carriers that are commonly used include lactose and corn starch. Lubricating agents, such as magnesium stearate, are also typically added. For oral administration in a capsule form, useful diluents include lactose and dried corn starch. When aqueous suspensions are administered orally, the active ingredient is combined with emulsifying and suspending agents. If desired, certain sweetening and/or flavoring and/or coloring agents may be added.

Administered in the form of suppositories for rectal administration is possible. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax, and polyethylene glycols.

Topical administration may be useful when the desired treatment involves areas or organs readily accessible by topical application. For application topically to the skin, a composition should be formulated with a suitable ointment containing the active components suspended or dissolved in a carrier. Carriers for topical administration of the compounds of this invention include, but are not limited to mineral oil, liquid petroleum, while petroleum, propylene glycol, polyoxyethylene or polyoxypropylene compound, emulsifying wax and water. Alternatively, compositions can be formulated with a suitable lotion or cream containing the active compound suspended or dissolved in a carrier. Suitable carriers include, but are not limited to, mineral oil, sorbitan monostearate, polysorbate 60, 2-octyldodecanol, benzyl alcohol and water. A composition may also be topically applied to the lower intestinal tract by rectal suppository formulation or in a suitable neat formulation. Topically-transdermal patches are also possible.

Administrating by nasal aerosol or inhalation is another aspect. Such compositions are prepared according to techniques well-known in the art of pharmaceutical formulation and may be prepared as solutions in saline employing benzyl alcohol or other suitable preservatives, absorption promoters to enhance bioavailability, fluorocarbons, and/or other solubilizing or dispersing is another aspect agents known in the art.

In principle, dosage levels of between about 0.01 and about 25 mg/kg body weight per day, preferably between about 0.5 and about 25 mg/kg body weight per day of the active ingredient compound are useful in the prevention and treatment of viral infection, including HIV infection. Typically, the compound (or a pharmaceutical composition) will be administered from about 1 to about 5 times per day or alternatively, as a continuous infusion. Such administration can be used as a chronic or acute therapy. The amount of active ingredient that may be combined with the carrier materials to produce a single dosage form will vary depending upon the patient treated and the particular mode of administration. A typical preparation will contain from about 5% to about 95% active compound (w/w).

As used herein patient in need of treatment refers to a mammal (e.g., a human).

Compounds according to a formula herein can be prepared by adapting a synthesis for making 5-oxopyrrolidine and indoles. Suitable starting materials having an indole moiety may be prepared in an analogous to a synthesis described in Synthesis, 2014, 46, 35-41, Synlett, 2012, 23, 2511-2515, and U.S. Pat. No. 2,496,163. As will be appreciated, additional compounds and analogs thereof within the formula herein can be synthesized by selecting appropriate starting materials and reactants.

In assessing a compound according to any of the formulas herein, an assay(s) can be conducted.

An HIV-1 assay can be (is) conducted. A present compound(s) and control compound(s) to be tested are obtained (made or have made). For instance, azidothymidine (AZT) a nucleoside reverse transcriptase inhibitor (NRTI) from Sigma Aldrich. Cell lines can be provided. For example, the Human embryonic kidney 293T cell (Hek293) and HeLa cell line were provided by Dr. Tshaka Cunningham. Mutagenized integrase encoding a fusion protein of enhanced green fluorescent protein with maltose binding protein (eGFP-MBP-IN-R262/3/6A and eGFP-MBP-IN-K236/240) as well as integrase wild type (eGFP-MBP-IN-WT) DNA plasmid and HIV-11entiviral vector encoding eGFP are obtained. In the present case, these were provided by Dr. Mark A. Museing (Rockefeller University).²⁵ All cell lines were maintained in RPMI 1640 medium supplemented with 10% fetal bovine serum and 1% penicillin and streptomycin. A compound represented by a formula herein that passes the HIV-1 assay is selected.

In assessing suitability of a compound according to any of the formulas herein, an integrase screening assay can be conducted. In the present case, an exemplary such assay is a cellular integrase assay. A cellular integrase assay can be performed in a 24 well plate. The assay is designed to test compounds in 3-fold serial dilutions (10, 3.3, 1.1 and 0.33 μM) with 0.1% DMSO. Negative and positive control included eGFP-MBP-IN-R262/3/6A, eGFP-MBP-IN-K236/240 and eGFP-MBP-IN-WT respectively supplemented with 0.1% DMSO. Refinement of the assay for optimal results is feasible. For example, refinement herein yields the following protocol. HEK 293T cells (˜0.4*10̂5 cells/ml) are seeded and incubated at 37° C. (5% CO₂) for ˜3 days on Poly-L-lysine treated glass coverslips until 80-90% confluent (˜3.2*10̂5 cells/ml). Next, cells are transfected with 1 μg of eGFP-MBP-INR262/3/6A, eGFP-MBP-IN-K236/240 and eGFP-MBP-IN-WT DNA plasmid simultaneously with test compounds, at varying concentrations mentioned above, using lipofectamine 2000 transfection reagent (μL) at a ratio of 1:2 DNA plasmid to lipofectamine. After 4 hrs of incubation (at 37° C., 5% CO₂), the lipofectamine mix is removed and replaced with fresh medium containing serum in the presence of the test compounds. The plate is incubated overnight at 37° C. (5% CO₂). Finally cells are treated again with fresh medium containing test compounds and incubated overnight at 37° C. (5% CO₂). After a day later (24 hours) cells are fixed with 4% paraformaldehyde in PBS and mounted in DAPI (Vectashield, Vector Laboratories) on glass slides for visualization. The slides are visualized and images, of at least 2 fields per glass disk, are prepared. Images may be prepared using an Olympus 1X51 fluorescence microscope equipped with a camera by way of example. This experiment can be repeated to ensure consistency of results.

In another aspect of assessing suitability of a compound according to any of formulas herein, a HIV-1 lentiviral vector screening assay can be performed. For example, to provide a measure of additional assurance the compounds identified are capable of exhibiting antiviral activity and are capable of permeating into the cells. For example, the HIV-1 lentiviral vector screening assay can be performed following the HIV assay and/or the integrase assay. For the vector assay HeLa cells can be utilized and the experiment can be performed in a suitable plate, such as a 24 well plate. In a manner similar to the integrase assay, the experiment can be designed to test further the compounds, such as compounds that passed another suitable assay, such as the HIV-1 screening assay or the integrase screening assay, with 2-fold serial dilution (60, 30, 15, 7.5 and 3.7 μM) in parallel with HIV-1 lentiviral vector and controls. A negative control, e.g., AZT, and a positive control as vector only and blank as un-infected are appropriately selected. Again, the concentration of DMSO was limited to 0.1%. Hela cells (˜3.2*10̂5 cells/ml) were infected with HIV-1 lentiviral vector in the presence of the compounds to be tested and controls. After the first 24 hours of incubation at 37° C. (5% CO₂), cells are replaced with fresh media containing test compounds and incubated for an additional 24 hrs. Cells are fixed with 4% paraformaldehyde in PBS and mounted in DAPI on glass slides for visualization and quantification. Images, of at least 2 fields per glass disk are obtained, such as images taken with an Olympus 1X51 fluorescence microscope equipped with a camera. EGFP fluorescence spanning an average area of 12*10̂5 square pixels is quantified using Image J software, from which IC₅₀ values can bee extrapolated using graph pad PRISM software.

It will be appreciated that in vitro testing, which may include the screening(s) described above, would be indicative of efficacy in a method of treating a patient or treating cells as described herein. Allosteric inhibitory activities are confirmed by the assay(s).

It will be appreciated that use of a compound herein for a method as described is unrelated to any method of treatment for a microbial infection. In other words, antiviral activity (here inhibition of HIV or inhibition of HIV integrase) would be unexpected even if a compound may exhibit antimicrobial activity or activity against another viral disease. A patient population herein constitutes patients in need of treatment against HIV, which includes a method for inhibition of HIV integrase.

Therefore in another of its aspects, as discussed above, the discovery includes any of the methods disclosed and further comprises providing a compound within the scope of the formulas is one that assayed as exhibiting antiviral activity (e.g., anti-HIV 1; inhibitor of IN), and using the compound in the method of treatment or in the method of inhibiting IN as described herein, particularly for the preferred patient population.

The amino acid sequence for IN is described in Crystral structure of the HIV-1 integrase catalytic core and C-terminal domains: a model for viral DNA binding, Proc. Natl. Acad. Sci., USA 97:8233-8238.

### Examples

The following non-limiting example(s) further describe(s) aspects of the present inventions.

In conducting the HIV assay, a representative test compound was obtained (TimTec) and azidothymidine (AZT) a nucleoside reverse transcriptase inhibitor (NRTI) were obtained from Sigma Aldrich. A Human embryonic kidney 293T cell (Hek293) and a HeLa cell line were obtained from Dr. Tshaka Cunningham. Mutagenized integrase encoding a fusion protein of enhanced green fluorescent protein with maltose binding protein (eGFP-MBP-IN-R262/3/6A and eGFP-MBP-IN-K236/240) as well as integrase wild type (eGFP-MBP-IN-WT) DNA plasmid and HIV-1 lentiviral vector encoding eGFP were obtained from Dr. Mark A. Museing (Rockefeller University). All cell lines were maintained in RPMI 1640 medium supplemented with 10% fetal bovine serum and 1% penicillin and streptomycin.

As an integrase screening assay, a cellular integrase assay was performed in a 24 well plate. The experiment was designed to test all compounds in 3-fold serial dilutions (10, 3.3, 1.1 and 0.33 μM) with 0.1% DMSO. Negative and positive control included eGFP-MBP-IN-R262/3/6A, eGFP-MBP-IN-K236/240 and eGFP-MBP-IN-WT respectively supplemented with 0.1% DMSO. Optimization of the assay resulted in the following protocol. HEK 293T cells (˜0.4*10̂5 cells/ml) were seeded and incubated at 37° C. (5% CO₂) for ˜3 days on Poly-L-lysine treated glass coverslips until 80-90% confluent (˜3.2*10̂5 cells/ml). Next, cells were transfected with 1 μg of eGFP-MBP-INR262/3/6A, eGFP-MBP-IN-K236/240 and eGFP-MBP-IN-WT DNA plasmid simultaneously with test compounds, at varying concentrations mentioned above, using lipofectamine 2000 transfection reagent (μL) at a ratio of 1:2 DNA plasmid to lipofectamine. After 4 hrs of incubation (@ 37° C., 5% CO₂), the lipofectamine mix was removed and replaced with fresh medium containing serum in the presence of the test compounds. Plate was incubated overnight at 37° C. (5% CO₂). Finally cells were treated again with fresh medium containing test compounds and incubated overnight at 37° C. (5% CO₂). 24 hours later cells were fixed with 4% paraformaldehyde in PBS and mounted in DAPI (Vectashield, Vector Laboratories) on glass slides for visualization. Slides were visualized and images, of at least 2 fields per glass disk, were taken with an Olympus 1X51 fluorescence microscope equipped with a camera. This experiment was repeated to ensure consistency of results.

A HIV-1 lentiviral vector screening assay was additionally performed after the integrase assay to help assure that a compound identified and selected exhibited antiviral activity and the capability to adequately permeate cells of interest (cells from or in a patient, such as a patient in need of treatment). For the vector assay HeLa cells were utilized and the HIV-1 lentiviral vector screening was performed in a 24 well plate. In a manner similar to the integrase assay, the HIV-1 lentiviral vector assay tested the compound(s) from the integrase assay, with 2-fold serial dilution (60, 30, 15, 7.5 and 3.7 μM) in parallel with HIV-1 lentiviral vector and controls. As the negative control AZT was used, a positive control as vector only and blank as un-infected were additionally used. Again the concentration of DMSO was limited to 0.1%. Hela cells (˜3.2*10̂5 cells/ml) were infected with HIV-1 lentiviral vector in the presence of the compound(s) tested and controls. After the first 24 hours of incubation at 37° C. (5% CO₂), cells were replaced with fresh media containing test compounds and incubated for an additional 24 hrs. Cells were fixed with 4% paraformaldehyde in PBS and mounted in DAPI on glass slides for visualization and quantification. Images, of at least 2 fields per glass disk were taken with an Olympus 1X51 fluorescence microscope equipped with a camera. EGFP fluorescence spanning an average area of 12*10̂5 square pixels was quantified using Image J software, from which IC₅₀ values were extrapolated using graph pad PRISM software.

Since the discovery of a potent nuclear localization signal (NLS) located on the C-terminal domain of HIV-1 integrase, mutagenesis experiments have identified amino acid residues 236/240 and 262/263/266 as essential for modulating HIV IN nuclear import.²⁵ With the aid of structure-based virtual screening equipped with the knowledge of two allosteric sites essential for HIV IN nuclear import, compounds of promise are identified. While not wishing to limit the scope of the present discoveries by a hypothesis, it appears from the present discoveries that small molecules can function as inhibitors by binding to certain amino acid residues in IN so as to block the IN, e.g., inhibit it from functioning. For example, a small molecule within the scope of formula (1), such as compound 209, that binds amino acid residues 236/240 or 262/263/266 will display a nuclear exclusion phenotype like or similar to that of mutant HIV-IN DNA plasmids eGFP-MBP-IN-R262/3/6A and eGFP-MBP-IN-K236/240.

For example, to illustrate the foregoing methodology, a presently preferred compound from amongst those tested, N-(3-ethoxy propyl)-1-[2-(5-methyl-1H-indol-3-yl)ethyl]-5-oxopyrrolidine (“209”), is representative and it exhibited inhibition against HIV to prevent nuclear import of the HIV integrase. This is indicative that a compound having efficacy as determined herein, such as by the screening methods, would be useful in a method for treating against HIV or HIV integrase. The data support the method for treating retroviral infections and more specifically inhibition of HIV infection comprises administering to an infected cell(s) or a human or mammal in need of treatment an effective amount of at least one anti-viral compound, in which at least one of the anti-viral compounds is N-(3-ethoxy propyl)-1-[2-(5-methyl-1H-indol-3-yl)ethyl]-5-oxopyrrolidine (“209”).

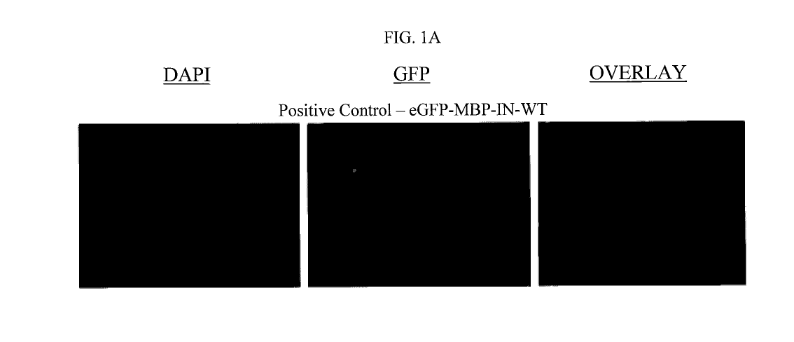

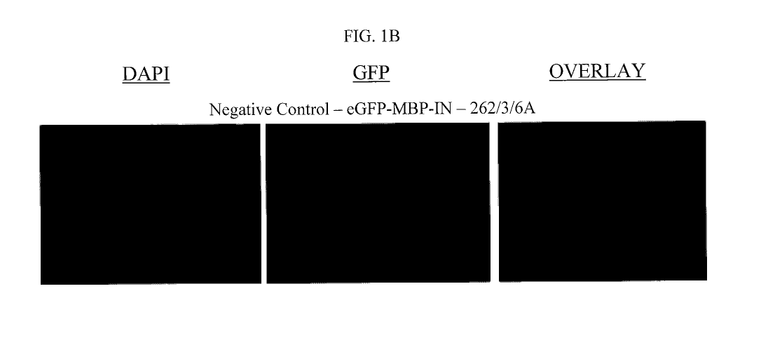

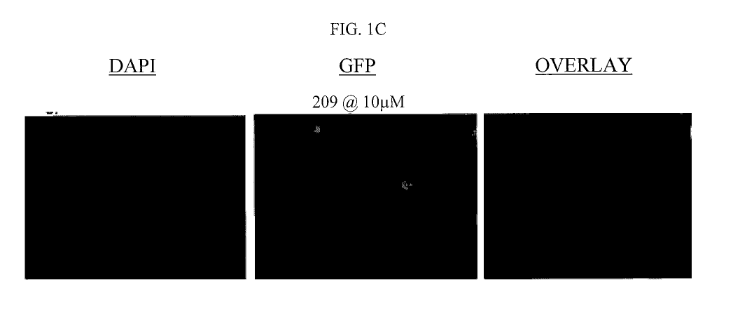

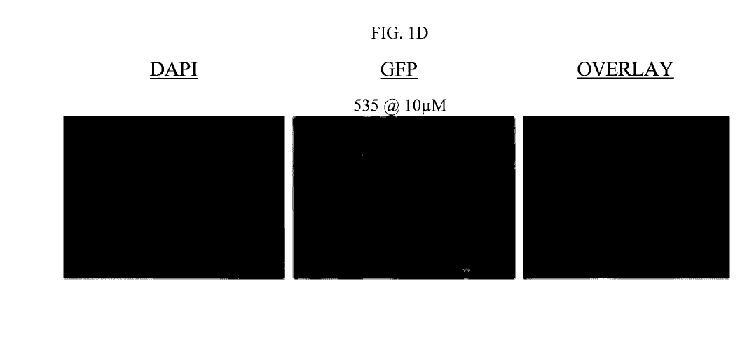

FIG. 1A clearly shows transfected HIV-1 IN accumulates within the nuclei of cells, while transfected NLS mutant integrase, eGFP-MBP-IN-R262/3/6A, was extranuclear with integrase mostly localized within the cytoplasm of cells (FIG. 1B). As shown in FIG. 1 the representative compound (“209”) depicted similar phenotypic results as that of eGFP-MBP-IN-R262/3/6A, which clearly demonstrates inhibition of nuclear import of IN. In FIGS. 1C and 1D, for cells expressing eGFP-IN, fluorescence is observed in the cytoplasmic region, confirmed by the DAPI staining of the nucleus (blue images) which is the obvious dark vacuoles in the GFP images.

The validation of the compound(s) as an inhibitor of HIV-1 IN nuclear import via a representative compound was seen in subsequent experimental validation of its effectiveness as an antiretroviral (ARV) and its ability to permeate the cells.

The advantage of IN screening assay is the plasma membrane of the cells was permeabilized using lipofectamine which perforates the cell membrane without compromising the integrity of the cell. As a result easy transport and deposition of both drug and fluorescent IN across the cell membrane into the cytoplasm is achieved. Lentiviral vector assay on the other hand does not involve transfection therefore allows for passive transport of test inhibitors across the cell membrane. HIV-1 lentiviral vector screening of the representative compound 209 and its combination of with AZT as the negative control was conducted. AZT exhibited remarkable antiviral activity in cell culture (IC50 0.23 μM) as expected. In comparison, the representative compound 209 (IC50 1.14 nM) exhibited micromolar and nanomolar activity respectively (FIG. 2).

FIG. 1 depicts the C-terminal domain of HIV-1 IN, nuclear import inhibition screening assay results. (HEK 293T cells transfected with HIV-1 integrase (C-terminal domain) DNA plasmid encoding enhanced green fluorescent fusion protein and maltose binding protein (eGFPMBP IN).)

In FIG. 1(A) the positive control, eGFP-MBP-IN-WT with no drug results in nuclear localization of IN is shown.

In FIG. 1(B) the negative control, eGFP-MBP-IN-R262/3/6A chimeric triple mutant exhibiting nuclear exclusion of the fusion protein is shown.

In FIG. 1(C) another test compound (“209”) with eGFP-MBP-IN-WT exhibiting similar phenotype as that of eGFP-MBP-IN-R262/3/6A that is, nuclear exclusion of the fusion protein.

In FIG. 1 (D) shows a different test compound with eGFP-MBP-IN-WT (for comparison) exhibiting again nuclear exclusion of the fusion protein in majority of cells.

FIG. 2 graphically presents the dose response curves, including a curve for a representative compound (“209”) and AZT. A HIV-1 lentiviral vector cell culture screening assay protocol was applied for testing antiviral activity of the test compounds. When comparing the effect of the representative test compound and the negative control AZT to HIV infectivity in the presence of no drug (Fluorescence Intensity 7.0*10⁷), all the test compounds exhibited an inhibitory effect in a dose dependent manor

Integration of HIV-1 viral genome into host DNA, located in the nucleus of cells, is an essential step for HIV infection. Nuclear import of integrase is impossible without the formation of pre-integration complex (PIC) which is recognized by members of the importin family and nuclear pore protein which transports PIC complex to the nucleus where integration occurs. With a complete understanding of the mechanism of action of IN and the evolution of N resistant mutations is still unfolding, it is postulated that IN may represent a target for new ARV drugs.

Novel integrase inhibitors have been discovered as described herein that bind to sites in IN to inhibit its activity such inhibitors include compounds that bind allosteric sites on the C-terminal domain of the protein.

Advantageously, there is no human homolog of IN enzyme, hence disrupting the function of IN should be tolerable, if not harmful, to human physiology. Therefore, a significant unexpected attribute of the present inventions lies in targeting residues that are less susceptible to mutation and are essential for integrase activity.

Advantageously, useful application of a present active ingredient that targets these regions according to a present method, which can contribute to lower HIV infection rates and increasing life expectancy of a patient.

Present indications are that HIV patients may be less likely develop resistance to the drug.

The results of the integrase screening assay clearly showed the representative compound displayed inhibition of nuclear import of IN similar to that of the triple mutant chimeric IN. However, further work showed this result translates to inhibition of HIV replication in cells from the standpoints of drug permeability and selectivity. For instance, results of the lentiviral vector screening indicate, without optimization, the representative compound clearly inhibited HIV-1 replication in a dose-dependent manor and at high concentrations without introducing cellular toxicity.

The complete disclosure of all the references cited hereinbelow is incorporated herein by reference.
1. UNAIDS UNAIDS report on the global AIDS epidemic.; Joint United Nations Programme on HIV/AIDS (UNAIDS): 12.
2. Menendez-Arias, L. Molecular basis of human immunodeficiency virus type 1 drug resistance: overview and recent developments. Antiviral Res. 2013, 98 (1), 93-120.
3. Archin, N. M.; Liberty, A. L.; Kashuba, A. D.; Choudhary, S. K.; Kuruc, J. D.; Crooks, A. M.; Parker, D. C.; Anderson, E. M.; Kearney, M. F.; Strain, M. C.; Richman, D. D.; Hudgens, M. G.; Bosch, R. J.; Coffin, J. M.; Eron, J. J.; Hazuda, D. J.; Margolis, D. M. Administration of vorinostat disrupts HIV-1 latency in patients on antiretroviral therapy. Nature 2012, 487 (7408), 482-485.
4. Archin, N. M.; Keedy, K. S.; Espeseth, A.; Dang, H.; Hazuda, D. J.; Margolis, D. M. Expression of latent human immunodeficiency type 1 is induced by novel and selective histone deacetylase inhibitors. AIDS 2009, 23 (14), 1799-1806.
5. Zhan, P.; Liu, X.; De, C. E. Blocking nuclear import of pre-integration complex: an emerging anti-HIV-1 drug discovery paradigm. Curr. Med. Chem. 2010, 17 (6), 495-503.
6. Brown, P. O. Integration; Outline of the Integration process. In Retroviruses, Coffin J M, Hughes S H, Varmus H E ed.; Cold Spring Harbor Laboratory Press: 1997.
7. Whittaker, G. R.; Kann, M.; Helenius, A. Viral entry into the nucleus. Annu. Rev. Cell Dev. Biol. 2000, 16, 627-651.
8. Ceccherini-Silberstein, F.; Malet, I.; D'Arrigo, R.; Antinori, A.; Marcelin, A. G.; Perno, C. F. Characterization and structural analysis of HIV-1 integrase conservation. AIDS Rev. 2009, 11(1), 17-29.
9. Quashie, P. K.; Mesplede, T.; Wainberg, M. A. Evolution of HIV integrase resistance mutations. Curr. Opin. Infect. Dis. 2013, 26 (1), 43-49.
10. FDA, FDA approval of Isentress (raltegravir). U.S. Food and Drug Administration (FDA). Jun. 25, 2009.
11. FDA, Approval of new fixed dose combination, Stribild (elvitegravir, cobicistat, emtricitabine, tenofovir disoproxil fumarate). U.S. Food and Drug Administration. Nov. 7, 2012.
12. Olin, J. L.; Spooner, L. M.; Klibanov, O. M. Elvitegravir/cobicistat/emtricitabine/tenofovir disoproxil fumarate single tablet for HIV-1 infection treatment. Ann. Pharmacother. 2012, 46 (12), 1671-1677.
13. Gilead, Gilead Receives Complete Response Letters from U.S. Food and Drug Administration for Elvitegravir and Cobicistat. Gilead Sciences, Inc., Apr. 30, 2013. Gilead.
14. Malet, I.; Delelis, O.; Valantin, M. A.; Montes, B.; Soulie, C.; Wirden, M.; Tchertanov, L.; Peytavin, G.; Reynes, J.; Mouscadet, J. F.; Katlama, C.; Calvez, V.; Marcelin, A. G., Mutations associated with failure of raltegravir treatment affect integrase sensitivity to the inhibitor in vitro. Antimicrob. Agents Chemother. 2008, 52 (4), 1351-1358.
15. Shimura, K.; Kodama, E.; Sakagami, Y.; Matsuzaki, Y.; Watanabe, W.; Yamataka, K.; Watanabe, Y.; Ohata, Y.; Doi, S.; Sato, M.; Kano, M.; Ikeda, S.; Matsuoka, M., Broad antiretroviral activity and resistance profile of the novel human immunodeficiency virus integrase inhibitor elvitegravir (JTK-303/GS-9137). 1 Virol. 2008, 82 (2), 764-774.
16. Malet, I.; Calvez, V.; Marcelin, A. G., The future of integrase inhibitors of HIV-1. Curr. Opin. Virol. 2012, 2 (5), 580-587.
17. Christ, F.; Voet, A.; Marchand, A.; Nicolet, S.; Desimmie, B. A.; Marchand, D.; Bardiot, D.; Van der Veken, N. J.; Van, R. B.; Strelkov, S. V.; De, M. M.; Chaltin, P.; Debyser, Z. Rational design of small-molecule inhibitors of the LEDGF/p75-integrase interaction and HIV replication. Nat. Chem. Biol. 2010, 6 (6), 442-448.
18. Christ, F.; Shaw, S.; Demeulemeester, J.; Desimmie, B. A.; Marchand, A.; Butler, S.; Smets, W.; Chaltin, P.; Westby, M.; Debyser, Z.; Pickford, C. Small-molecule inhibitors of the LEDGF/p75 binding site of integrase block HIV replication and modulate integrase multimerization. Antimicrob. Agents Chemother. 2012, 56 (8), 4365-4374.
19. Wang, J. Y.; Ling, H.; Yang, W.; Craigie, R. Structure of a two-domain fragment of HIV-1 integrase: implications for domain organization in the intact protein. EMBO J., 2001, 20 (24), 7333-7343.
20. Chen, J. C.; Krucinski, J.; Miercke, L. J.; Finer-Moore, J. S.; Tang, A. H.; Leavitt, A. D.; Stroud, R. M. Crystal structure of the HIV-1 integrase catalytic core and C-terminal domains: a model for viral DNA binding. Proc. Natl. Acad. Sci. U S. A 2000, 97 (15), 8233-8238.
21. McColl, D. J.; Chen, X., Strand transfer inhibitors of HIV-1 integrase: bringing IN a new era of antiretroviral therapy. Antiviral Res. 2010, 85 (1), 101-118.
22. Zheng, R.; Jenkins, T. M.; Craigie, R., Zinc folds the N-terminal domain of HIV-1 integrase, promotes multimerization, and enhances catalytic activity. Proc. Natl. Acad. Sci. U S. A 1996, 93 (24), 13659-13664.
23. Engelman, A.; Craigie, R., Identification of conserved amino acid residues critical for human immunodeficiency virus type 1 integrase function in vitro. J. Virol. 1992, 66 (11), 6361-6369.
24. Lu, R.; Ghory, H. Z.; Engelman, A., Genetic analyses of conserved residues in the carboxyl-terminal domain of human immunodeficiency virus type 1 integrase. J. Virol. 2005, 79 (16), 10356-10368.
25. Muesing, M. A.; Cunningham, T. J., Nuclear localization signal of lentiviral integrase and methods of use thereof. US 20060134651A1, Jun. 22, 2006.
26. Wiskerchen, M.; Muesing, M. A. Human immunodeficiency virus type 1 integrase: effects of mutations on viral ability to integrate, direct viral gene expression from unintegrated viral DNA templates, and sustain viral propagation in primary cells. J. Virol. 1995, 69 (1), 376-386.
27. Mousnier, A.; Leh, H.; Mouscadet, J. F.; Dargemont, C. Nuclear import of HIV-1 integrase is inhibited in vitro by styrylquinoline derivatives. Mol. Pharmacol. 2004, 66 (4), 783-788.
28. Levin, A.; Armon-Omer, A.; Rosenbluh, J.; Melamed-Book, N.; Graessmann, A.; Waigmann, E.; Loyter, A. Inhibition of HIV-1 integrase nuclear import and replication by a peptide bearing integrase putative nuclear localization signal. Retrovirology. 2009, 6, 112.

What is claimed is:

1. A method for inhibiting HIV integrase in target cell(s) or in a patient comprises administering to said target cell(s) or to a patient in need of treatment an effective amount of at least one anti-viral compound, wherein the anti-viral compound comprises a compound having an N-indol heteroarylcarboxamide scaffold which is represented by the formula: 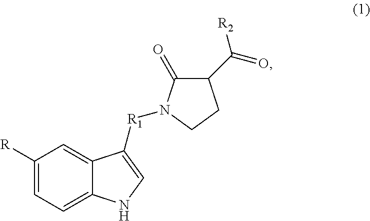
wherein, independent of each other,
R independently represents hydrocarbyl, halogeno, amino, substituted amino, or alkoxy, wherein substituted amino is represented by —NR₃R₄ wherein R₃ and R₄, are not both hydrogen and independently represent alkyl or alkenyl,
R₁ represents di-valent hydrocarbyl, substituted or unsubstituted, and
R₂ represents an ether moiety —R₅—O—R₆ wherein R₅ and R₆ are alkyl moieties.

2. The method for inhibiting HIV integrase according to claim 1, wherein R represents lower alkyl.

3. The method for inhibiting a HIV integrase according to claim 2, wherein R represents C₁-C₆ alkyl.

4. The method for inhibiting HIV integrase according to claim 1, wherein R represents halogeno.

5. The method for inhibiting HIV integrase according to claim 1, wherein R represents amino.

6. The method for inhibiting a HIV integrase according to claim 1, wherein R represents —NR₃R₄ wherein R₃ and R₄, are not both hydrogen and independently represent alkyl or alkenyl.

7. The method for inhibiting HIV integrase according to claim 1, wherein R represents alkoxy.

8. The method for inhibiting HIV integrase according to claim 1, wherein R₁ is a di-valent alkylene group.

9. The method for inhibiting HIV integrase according to claim 4, wherein R₁ represents a di-valent alkylene group having one to seven carbon atoms.

10. The method for inhibiting HIV integrase according to claim 8, wherein R₁ represents —(CH₂)₃—.

11. A method for inhibiting HIV integrase according to claim 1, wherein R₁ is a di-valent aryl group.

12. The method for inhibiting HIV integrase according to claim 11, wherein R₁ is phenylene.

13. The method for treating against HIV by inhibiting HIV integrase according to claim 1, wherein, independently of each other, R₅ and R₆ represent an alkyl moiety having one to ten carbon atoms.

14. The method for inhibiting HIV integrase according to claim 1, wherein the compound is N-(3-ethoxy propyl)-1-[2-(5-methyl-1H-indol-3-yl)ethyl]-5-oxopyrrolidine.

15. The pharmaceutical composition formulated for inhibiting HIV integrase comprising
at least one compound having an N-indol heteroarylcarboxamide scaffold as an effective ingredient, said at least one compound is represented by the formula: 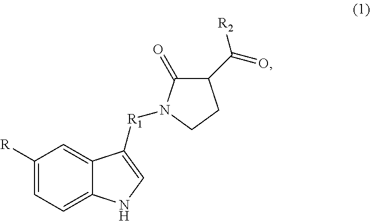
wherein, independent of each other,
R independently represents hydrocarbyl, halogeno, amino, substituted amino, or alkoxy, wherein substituted amino is represented by —NR₃R₄ wherein R₃ and R₄, are not both hydrogen and independently represent alkyl or alkenyl,
R₁ represents di-valent hydrocarbyl, substituted or unsubstituted, and
R₂ represents an ether moiety —R₅—O—R₆ wherein R₅ and R₆ are alkyl moieties; and a pharmaceutically acceptable adjuvant.

16. The pharmaceutical composition according to claim 14, wherein R₁ is a di-valent C₁-C₇ alkylene moiety.

17. A novel compound having an N-indol heteroarylcarboxamide scaffold selected from those represented by formula: 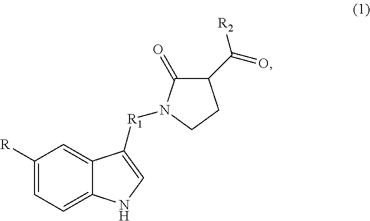
wherein, independent of each other,
R independently represents hydrocarbyl, halogeno, amino, substituted amino, or alkoxy, wherein substituted amino is represented by —NR₃R₄ wherein R₃ and R₄, are not both hydrogen and independently represent alkyl or alkenyl,
R₁ represents di-valent hydrocarbyl, substituted or unsubstituted, and
R₂ represents an ether moiety —R₅—O—R₆ wherein R₅ and R₆ are, independent of each other, alkyl or aryl moieties.

18. The novel compound according to claim 16, wherein R represents C₁-C₆ alkyl, halogeno, or a C₁-C₃ alkoxy; R₁ represents phenylene or a divalent C₁-C₇ alkylene moiety; and R₂ represents an ether moiety —R₅—O—R₆ wherein R₅ and R₆, independent of each other, represent a C₁-C₆ alkyl moiety.
